(19) United States
(12) Patent Application Publication
(10) Pub. No.: US 2009/0324717 A1
(43) Pub. Date: Dec. 31, 2009

(54) Extended release pharmaceutical formulation of metoprolol and process for its preparation

(75) Inventors: Josep M. Suñe Negre, Barcelona (ES); Maria Del Carmen Vall Pares, Barcelona (ES); Noemi Alvarez Casares, Sant Cugat del Valles (Barcelona) (ES); Francisco Gual Pujol, Barcelona (ES)

Correspondence Address: BERENBAUM WEINSHIENK PC, 370 17TH STREET, SUITE 4800, DENVER, CO, 80202, US

(73) Assignee: FARMAPROJECTS, S. A., L'Hospitalet de Llobregat (Barcelona) (unknown)

(21) Appl. No.: 12/375,524

(22) PCT Filed: Jul. 26, 2007
(86) PCT No.: PCT/EP2007/057715

§ 371 Date: Jan. 28, 2009

(30) Foreign Application Priority Data
Jul. 28, 2006 (EP) EP 06118059.2

(51) Int. Cl. A61K 9/26; A61K 9/50; A61K 31/138

(52) U.S. Cl. 424/470; 424/490; 424/494; 424/495; 424/497; 424/498; 514/652

(57) ABSTRACT

The invention provides an extended release coated granule comprising a granule having a particle size ranging from 0.2 to 2 mm, a friability lower than or equal to 1% and comprising metoprolol succinate as active ingredient in an amount ranging from 10 to 75% by weight of the granule and at least one binder selected from microcrystalline cellulose and methylcellulose, coated with a film-former coating agent. It also provides a process for the preparation of said extended release coated granules, as well as pharmaceutical formulations containing them.

The present invention relates to extended release coated granules of metoprolol succinate, a process for their preparation and their uses in extended release pharmaceutical formulations. It also relates to specific extended release coated granules which could be useful with other active ingredients.

### BACKGROUND OF THE INVENTION

Metoprolol succinate is the international nonproprietary name (INN) of (±) 1-(isopropylamino)-3-[p-(2-methoxyethyl) phenoxy]-2-propanol succinate (2:1) and corresponds to formula:

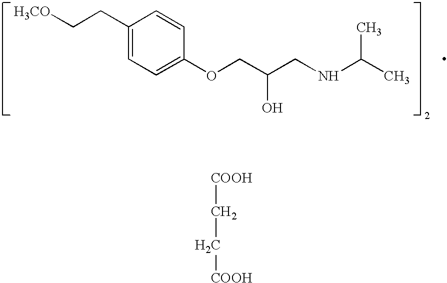

Metoprolol is a beta₁-selective (cardioselective) adrenoreceptor blocking agent. Metoprolol succinate is useful in the treatment of cardiovascular diseases such as hypertension, angina pectoris, stabilized symptomatic mild to severe chronic heart failure, tachyarrhythmias, especially supraventricular tachycardias, in maintenance treatment after myocardial infarction, functional heart disorders with palpitations and in migraine prophylaxis. In the medical treatment of these types of diseases it is advantageous to have a constant or sustained drug concentration in the blood.

The concept of extended release formulations was developed to reduce the number of daily drug administrations, particularly for those drugs requiring reasonably constant blood levels over a long period of time. Extended release formulations have also been adopted for those drugs that need to be administered at high doses, but are likely to cause undesirable side effects by a fast release of the drug.

Extended release formulations containing a metoprolol salt as active ingredient are known in the state of the art. Said formulations comprise as the extended release agent, for example, an ion-exchange resin (EP 560816), an osmotic system (EP 723435-A1, EP 1455751-A1, EP 1469834-A1, EP 1499295-A1 and WO 2004069234), a salt of alginic acid as a polymer (GB 2207353-A) or a modified polysaccharide (EP 1322293), among others.

European patent application EP 293347 describes, for the first time, metoprolol succinate and an oral pharmaceutical composition which comprises a core containing a therapeutically active compound coated with a layer comprising a) 10 to 85% by weight of an anionic polymer soluble at a pH above 5.5, and b) 15 to 90% by weight of a water-insoluble polymer selected from quaternary ammonium-substituted acrylic polymers.

European patent application EP 277127 discloses controlled release beads of active compounds, including metoprolol succinate (Example 9), coated with a membrane controlling the drug release. The beads contain one or more pharmaceutically active compounds applied on a compact insoluble core material with a porosity of less than 15%, whereby the active compound forms a compact layer on the insoluble core and this compact layer is further covered with a release controlling polymeric membrane.

European patent application EP 220143 discloses controlled release preparations of salts of metoprolol. Particularly, Example 3 discloses a formulation of metoprolol succinate in the form of pellets constituted only by metoprolol succinate, having an average particle size of 0.42 mm and coated with a solution containing ethylcellulose, hydroxypropylmethylcellulose, acetyltributylcitrate, methylene chloride and isopropylic alcohol.

International application WO 2006048895 discloses a platform for use with any active compound based on the use of stable aqueous dispersions of wax or combination of waxes for coating. Example 15 discloses the use of aqueous wax coating dispersions to retard the release of metoprolol succinate pellets. Metoprolol succinate (80% w/w) and microcrystalline cellulose were mixed. An aqueous solution of povidone was added and mixed. The mixture was extruded, spheronized and the pellets obtained were dried and coated with an aqueous wax coating. Coated pellets were filled into capsules and showed a slightly retarded dissolution profile at pH 6.8 (at 1 h 65.3% of metoprolol succinate was released and 77.1% at 2 h). Granules obtained with the same composition as that of the pellets were not suitable for coating.

In spite of the existence of extended release preparations of metoprolol or its salts, there is a need for alternative extended release forms of the metoprolol succinate salt that modulate the release rate of the drug, in order to maintain therapeutic activity while reducing side effects.

### SUMMARY OF THE INVENTION

Extended release compositions of highly water-soluble drugs are more difficult to formulate because a sudden release, also called dose-dumping effect, is usually found with these drugs. It is known that metoprolol succinate is very soluble in aqueous media, and this high solubility is a critical point when formulating an extended release composition comprising metoprolol succinate.

The present invention relates to extended release coated granules of metoprolol succinate.

The term “granule” is to be understood in the present invention as the direct result of granulation processes, either by wet or dry granulation techniques. Wet granulation is preferred for the granules of the invention. By their nature, granules have an irregular form as opposed to pellets and beads. Pellets and beads, obtained by extrusion-spheronization techniques or from the sequential coating of spheroidal cores, are almost spherical. Although pellets are sometimes referred to as spherical granules, pellets are not an object of the present invention.

The present inventors have found that the presence of at least one binder selected from microcrystalline cellulose and methylcellulose in the granules plus the fact that the granules have a friability lower than or equal to 1% facilitate the coating of such granules. These features are advantageous since they confer an appropriate hardness to the granules of the invention. A suitable hardness is important, as it will prevent breakage of the granules during the coating process. This is especially important because granules have an irregular form and are therefore more liable to breakage than pellets and beads during the coating process to provide extended release coatings and, in the case of tablets, also during the compression step.

An additional advantage of the granules of the invention is the fact that no extrusion-spheronization steps have to be carried out in order to obtain the granules of the invention as described for the preparation of pellets in some prior art documents, which also makes this process much simpler.

Furthermore, another advantage of the granules of the invention is that an extended release profile of the product can be achieved by coating the granules with a single coating layer, there being no need for additional coatings. Consequently, the process for the preparation of the coated granules is simpler and easier since less coating steps are needed.

Thus, a first aspect of the present invention is to provide an extended release coated granule consisting of a granule having a particle size ranging from 0.2 to 2 mm, a friability lower than or equal to 1% and comprising metoprolol succinate as active ingredient in an amount ranging from 10 to 75% by weight of the granule and at least one binder selected from microcrystalline cellulose and methylcellulose, said granule being coated with a film-former coating agent.

Without being bound by theory, metoprolol succinate release rate is believed to be determined by its diffusion through the micropores formed by the film-former coating agent. In fact, the release of the drug involves a combination of dissolution and diffusion effects: first water comes into contact with the granule through the micropores and dissolves the drug present inside the granule; the dissolved drug is then released from the granule. Advantageously, the diffusion of metoprolol succinate through the micropores of the coating, which is usually insoluble, gives rise to an extended release of the drug once it has been ingested.

WO 2006048895 addresses the problem of using aqueous dispersions of waxes for coating pharmaceutical compositions comprising any drug. Among the possible uses of such coatings, taste masking, stabilization and release modification are included. Release modification includes sustained, pulsatile, delayed or targeted release. Use of waxes as coating agents was restricted due to the need to use organic solvents or hot melt methods. Several drugs were used in the examples such as metformin HCl, metoprolol succinate, tibolone, phenyloin sodium, ursodiol, cetirizine HCl and pseudoephedrine HCl. This document does not disclose granules comprising metoprolol succinate as defined above. Instead, pellets are described after an extrusion-spheronization process in Example 15. The present invention does not concern pellets, but granules having specific properties. In a comparative example (see Example 6 below), granules were prepared having the same composition as the pellets described in Example 15 of WO 2006048895. After sieving the granules to obtain granules with a particle size in the range of 0.2 to 2 mm, the friability of such granules was found to be of 43% and therefore not suitable for the coating process required by the invention.

In WO 2006048895 pellets were filled into capsules, therefore they were not subjected to a demanding compression process which would require the pellets to have a high hardness value. On the other hand, the present invention is restricted to granules having a friability lower than or equal to 1% and an amount of metoprolol succinate ranging from 10 to 75% by weight of the granule before being coated, while the composition of Example 15 contains about 79.2% of metoprolol succinate in the uncoated pellets.

A second aspect of the invention is to provide a process for the preparation of the extended release coated granules of the invention which comprises the following steps: a) granulating a mixture comprising metoprolol succinate and at least one binder selected from microcrystalline cellulose and methylcellulose, and wherein the resulting amount of metoprolol succinate in the dry granules is comprised between 10 and 75% by weight; b) drying the granules resulting from step (a) if required; c) sieving the dried granules through a sieve with a mesh size of 1-2 mm; and then through a sieve with a mesh size of 0.2-0.4 mm in order to separate the granules with a size lower than the mesh size used; and d) coating the dried granules resulting from step (c) with a dispersion of a film-former coating agent.

With this process coated granules are obtained without agglomeration problems. Furthermore, the hardness of the coated granules obtained is sufficient for an efficient tabletting process, i.e. granules do not break during the compression step.

Furthermore, said process does not require complex or special equipment in order to prepare the granules of the present application, as compared to the process for preparing pellets or beads. Consequently, it is a cheaper alternative process with respect to other processes known in the state of the art.

A third aspect of the present invention is to provide an extended release pharmaceutical composition comprising coated granules as defined above together with appropriate amounts of pharmaceutical excipients or carriers.

A fourth aspect of the present invention is a process for preparing extended release pharmaceutical compositions, comprising: a) mixing the extended release coated granules as defined above with appropriate amounts of pharmaceutical excipients or carriers; b) compressing the mixture resulting from step (a); and c) optionally, coating the tablet cores resulting from step (b) with a coating dispersion which comprises at least one film-former compound.

Also part of the present invention is the use of the coated granules of metoprolol succinate defined above for the preparation of a medicament for the treatment of a cardiovascular disease, such as angina pectoris.

The invention also relates to a method of treatment and/or prophylaxis in patients, suffering from or susceptible to cardiovascular diseases such as angina pectoris, said method comprising the administration to said patients of a therapeutically effective amount of the pharmaceutical formulation comprising the extended release coated granules of metoprolol succinate of the present invention together with pharmaceutically acceptable diluents or carriers.

The inventors have found that some specific granule compositions developed for metoprolol succinate according to the present invention are also useful for the preparation of extended-release granules with other active ingredients, keeping the same advantageous properties that those pointed out above for the metoprolol succinate granules (e.g., appropriate hardness to carry out a coating process). Thus, it is possible to prepare coated granules containing other active ingredients and having an extended release profile. Therefore, in a further aspect, the invention relates to an extended release coated granule consisting of a granule having a particle size ranging from 0.2 to 2 mm, a friability lower than or equal to 1% and comprising an active ingredient in an amount ranging from 1 to 75% by weight, preferably from 10 to 75%, microcrystalline cellulose, methylcellulose, starch and optionally a wetting agent, preferably glycerol, said granule being coated with a film-former coating agent, preferably ethylcellulose.

The specific composition of such granules is suitable to confer extended release properties to a variety of active ingredients and offers a suitable alternative way to formulate extended release compositions. Some processes described in the invention for metoprolol succinate are useful for many other active ingredients and provide a simple way of preparing extended release granules which could be incorporated into extended release compositions. Thus, another aspect of the invention is a process for the preparation of such extended release coated granules comprising the steps of: a) granulating a mixture comprising an active ingredient, microcrystalline cellulose, methylcellulose and a solution of starch, and wherein the resulting amount of active ingredient in the dry granules is between 1 and 75% by weight; b) drying the granules resulting from step (a); c) sieving the dried granules through a sieve with a mesh size of 1-2 mm; and then through a sieve with a mesh size of 0.2-0.4 mm in order to separate the granules with a size lower than the mesh size used; and d) coating the dried granules resulting from step (c) with a dispersion of a film-former coating agent

Another aspect of the invention relates to extended release compositions comprising the above granules that comprise at least one active ingredient and to processes for the preparation of such compositions which include adding appropriate amounts of pharmaceutical excipients or carriers, optionally compressing such mixtures and optionally coating the compressed forms.

### DETAILED DESCRIPTION OF THE INVENTION

In the present invention the term “extended-release” is to be understood as defined in the United States Pharmacopeia 26, under the General Information section: “extended-release tablets are formulated in such manner as to make the continued medicament available over an extended period of time following ingestion”. Extended release is achieved by special formulation design and/or manufacturing method.

In the present invention the term “dispersion” is to be understood as a mixture in which fine particles of one substance are scattered throughout another substance, in the invention this other substance is a solvent. Dispersions include suspensions, colloids and solutions.

Parameters associated with the granules or granulates of the invention such as particle size, particle size distribution, friability and percentage of metoprolol, unless otherwise stated, refer to the granules arising from the granulation process, i.e., before the coating process.

Friability is the degree to which a solid is friable. A solid is friable when it can be easily crumbled into powder or small particles. Granule and tablet surfaces may be damaged and/or show evidence of lamination or breakage when subjected to mechanical shock or attrition and these effects are related with the friability of granules and tablets. Friability is determined according to an adaptation of the method described in European Pharmacopeia version 5.0, 2.9.7., weighing 10 g of the granulate.

The dissolution profile of the coated tablets of the invention is determined by the method described in USP 26 monograph for metoprolol succinate extended release tablets.

The particle size and particle size distribution of the granules of the invention are determined by sieving them through screens with specific mesh sizes.

In one embodiment of the first aspect of the invention the granule has a particle size distribution ranging from 0.2 to 2 mm and a friability lower than 1%. This range allows a more homogeneous release profile of the compositions comprising such granules, which is advantageous to prepare pharmaceutical compositions.

In another embodiment of the first aspect of the invention the granule has a particle size ranging from 0.2 to 1 mm.

In yet another embodiment of the first aspect of the invention, the amount of metoprolol succinate in the granule ranges from 40 to 75% by weight, more particularly 40 to 60% by weight.

In yet another embodiment of the first aspect of the invention, microcrystalline cellulose and methylcellulose are used as binders.

Commercially available fillers provide better flow properties to the blend before compression. The filler also provides cohesiveness to the tablet. Too little filler will result in flow problems and decrease hardness; too much filler may adversely affect the tablet size.

Yet another embodiment of the first aspect of the present invention is that the coated granule further comprises one or more binders selected from the group consisting of maize starch; gelatin; povidones; arabic gum; tragacanth gum; pectin; dextrin; glyceryl behenate; alginates; mannitol; lactose; hydroxyethylcellulose and its derivatives; hydroxyethylmethylcellulose and its derivatives; hydroxypropylcellulose and its derivatives; hydroxypropylmethylcellulose and its derivatives; bicalcium phosphate; tricalcium phosphate; lactose-povidone complexes; lactose-colloidal silica dioxide; liposaccharide-alkaline earth orthophosphate salt complexes; calcium carbonate and its derivatives; and calcium carbonate co-processed mixtures of calcium carbonate with sorbitol, mannitol, any other kind of saccharides, polysaccharides, copolyvidones, dextrins, maltodextrins, carboxymethylcelluloses, pregelatinized starch, cyclodextrins, cellulose ether, calcium gluconates, or calcium gluconates-lactates. Preferably the coated granule of the invention further comprises starch, more preferably maize starch as a binder. Starch confers a high hardness value to the granules and is especially suitable for the granule of the invention.

Yet another embodiment of the first aspect of the invention is that the film-former coating agent is selected from the group consisting of ethylcellulose; mono-, di- or triglycerides; fatty acids; waxes; synthetic mixed glycerides; hydrophilic cellulose derivatives with medium or high viscosity; polyvinyl acetates and chlorides; calcium phosphates and sulphates; hydrocolloids, hydrogels, methacrylic polymer compounds and derivatives; cellulose aceto-phthalates; cellulose hydrogen phthalates; and alginic acid derivatives. Preferably the film-former coating agent is ethylcellulose. Due to the high solubility of metoprolol succinate, ethylcellulose is particularly suitable in order to achieve a pronounced sustained release profile of the drug. Other film-former coating agents, do not achieve such a pronounced sustained release effect. Furthermore, ethylcellulose coating is flexible and does not break on compression.

It should be borne in mind that the final dosage forms typically contain drug loadings that are sufficiently high to cause problems if the entire dose is released quickly. This phenomenon, commonly called “dose-dumping”, can be avoided if sufficient coating is applied uniformly across the surface of the material that is to be coated.

In one embodiment of the second aspect of the invention, the granulation of step (a) further comprises the addition of a binding solution comprising at least one binder.

The coating of step (d) is preferably carried out using an amount of film-former coating agent ranging from 1 to 20% by weight in an appropriate solvent system resulting in a weight increase of between 10 and 40%; using fluid bed equipment.

In a further embodiment of the second aspect of the invention, the coating of step (d) results in a weight increase of the granule of between 20 and 30%. Preferably, the coating of step (d) results in a weight increase of 25%.

In another embodiment of the second aspect of the invention the binding solution is a starch paste comprising a solution of maize starch in a mixture of glycerol and water.

In yet another embodiment of the second aspect of the invention, step (b) is carried out at a temperature of between 30 and 70° C.

In yet another embodiment of the second aspect of the invention the film-former coating agent is dissolved in a solvent selected from the group consisting of ethanol, isopropylic alcohol, acetone, methylene chloride, water and mixtures thereof.

Solvents perform an important function in the film-coating process, since they aid in the application of the coating to the surface of the substrate. Good interaction between solvent and film-forming coating agent is necessary to ensure that optimal film properties are obtained when the coating dries. Another important function of solvent systems is to ensure that the film-forming coating agent is deposited onto the surface of the substrate in a controlled manner so that a coherent and adherent film coating is obtained.

In one embodiment of the third aspect of the invention the extended release pharmaceutical formulation comprises at least 90% by weight of metoprolol succinate as coated granules of the first aspect of the invention and up to 10% by weight of metoprolol succinate as uncoated granules with a particle size not greater than 0.4 mm, together with appropriate amounts of pharmaceutical excipients or carriers.

Preferably, the extended release pharmaceutical formulation comprises 95% by weight of metoprolol succinate as coated granules and 5% by weight of metoprolol succinate as uncoated granules.

In another embodiment of the third aspect of the invention the extended release pharmaceutical formulation is a tablet.

The film-former compound used for coating the tablet can be selected from hydroxypropylmethylcellulose, hydroxypropylcellulose or its derivatives, polyethylenglycoles, povidones and its derivatives, metacrylic polymeric compounds and derivatives, medium or high cellulosic derivatives, waxes, hydrocolloids, hydrogels and mixtures thereof, among others.

As mentioned above, it is also part of the invention the extended release coated granule consisting of a granule having a particle size ranging from 0.2 to 2 mm, a friability lower than or equal to 1% and comprising an active ingredient in an amount ranging from 1 to 75% by weight, preferably from 10 to 75%, microcrystalline cellulose, methylcellulose, starch and optionally a wetting agent, preferably glycerol, said granules being coated with a film-former coating agent, preferably ethylcellulose. Illustrative non-limitative examples of active ingredients are those listed in Martindale, The Extra Pharmacopoeia, 35th Ed., and in US Patent application US20070116729, pages 4-16, paragraphs 29 to 31, the disclosure of which is incorporated herein by reference in its entirety. Preferably, the active ingredient is selected from quetiapine and its salts, especially quetiapine fumarate, pramipexole and its salts, especially its dihydrochloride monohydrate, tolterodine and its salts, especially the tartrate salt.

Additional objects, advantages and features of the invention will become apparent to those skilled in the art upon examination of the description or may be learned by practice of the invention. Throughout the description and claims the word “comprise” and variations of the word are not intended to exclude other technical features, additives, components or steps. The disclosure in the abstract of this application is incorporated herein as reference. The following examples and drawings are provided by way of illustration, and they are not intended to be limiting of the present invention.

### EXAMPLES

### Example 1

| | Ingredient | Amount (mg) |
|---|---|---|
| Granules | Metoprolol succinate | 190.00 |
| | (72.6% w/w of granule) | |
| | Microcrystalline cellulose PH 101 | 63.45 |
| | Ethylcellulose N100 | 8.12 |
| | Granules | 261.57 |
| Coated granules | Triethylcitrate | 8.63 |
| | Eudragit ® RS 30D (solid fraction) | 73.50 |
| | Coated granules | 343.70 |
| Tablet | Coated granules | 343.70 |
| | Uncoated granules | — |
| | Croscarmellose sodium | 10.31 |
| | Talc | 27.50 |
| | Magnesium stearate | 6.87 |
| | Prosolv ® HD90 | 311.62 |
| | Tablet | 700.00 |
| Coated Tablet | Film coating suspension: | 10.50 |
| | Sepifilm ® 752 white | |
| | Coated tablet | 710.50 |

Eudragit ® RS 30D is an Eudragit dispersion with 30% solid fraction, Eudragit being an ammonio methylacrylate copolymer Type B.
Prosolv ® HD90 is microcrystalline cellulose 98% and colloidal anhydrous silica 2%.
Sepifilm ® 752 white is a film-coating suspension comprising 35-45% of hydroxypropylmethylcellulose, 27-37% talc, 15-25% of titanium dioxide and 5-10% of polyethylenglycol.

### Method of Preparation:

Batch size: 4000 tablets

### a) Granulation:

836 g of metoprolol succinate and 279.2 g of microcrystalline cellulose were sieved through a sieve with a 2 mm mesh and then blended in a double-cone blender for 10 minutes at 25 rpm. In a suitable container, fitted with an anti-combustion stirrer, 35.7 g of ethylcellulose were dissolved in an isopropanol (286 ml)/acetone (428 ml) mixture. The powder blend was placed in a double sigma blender and was mixed with the ethylcellulose solution until a mass with a suitable appearance and plasticity was obtained. The resulting mixture was screened in a wet granulator fitted with a 3 mm mesh screen. Finally, the granulate was dried in a forced air anti-combustion oven at 40° C. for 2 hours, sieved through an oscillating sieve fitted with a 1.2 mm mesh and then sieved through a vibrating sieve with a 0.355 mm mesh to separate the fine fraction. Until its subsequent use, the granulate was stored in a covered container. The friability of the granulate with a particle size comprised between 1.2 and 0.355 mm is 0.30%.

### b) Coating of the Granules:

Separately, in an appropriate glass container fitted with a stirrer, 34.5 g of triethylcitrate were added to 980 g of Eudragit® RS 30D. Then the dispersion was sieved through a sieve with a 0.1 mm mesh, stirring constantly. The granulate was placed in a fluid bed coater in order to coat it with the coating dispersion. The weight increase after the coating process was 31.4%.

### c) Compression:

41 g of croscarmellose, 110 g of talc, 27.5 g of magnesium stearate and 1246.5 g of Prosolv® HD90 were added to the coated granulate. The resulting mixture was compressed in suitable equipment in order to obtain tablets with a weight of 700 mg.

### d) Coating of Tablets:

The tablets resulting from the previous step were coated with Sepifilm® 752 white, until a 1.5% of weight increase was achieved. The dissolution profile of the obtained formulation is given below:

| Time (h) | % Dissolved |
|---|---|
| 1 | 14.3 |
| 4 | 48.2 |
| 8 | 55.1 |
| 20 | 59.7 |

### Example 2

| | Ingredient | Amount (mg) |
|---|---|---|
| Granules | Metoprolol succinate | 190.00 |
| | (46.2% w/w of granule) | |
| | Microcrystalline cellulose PH 101 | 82.40 |
| | Methylcellulose | 102.90 |
| | Povidone K 29-32 | 27.70 |
| | Soya lectin | 8.00 |
| | Granules | 411.00 |
| Coated granules | Ethylcellulose N100 | 57.70 |
| | Coated granules | 468.70 |
| Tablet | Coated granules | 468.70 |
| | Uncoated granules | — |
| | Microcrystalline cellulose PH 101 | 341.30 |
| | Microcrystalline cellulose PH 102 | 80.00 |
| | Magnesium stearate | 10.00 |
| | Tablet | 900.00 |
| Coated Tablet | Film coating suspension: | 13.50 |
| | Sepifilm ® 752 white | |
| | Coated tablet | 913.50 |

### Method of Preparation:

Batch size: 2330 tablets

### a) Granulation:

500 g of metoprolol succinate, 217 g of microcrystalline cellulose and 271 g of methylcellulose were sieved through a sieve with a 2 mm mesh and then blended in a double-cone blender for 10 minutes at 25 rpm. 73 g of povidone were dissolved in water in a suitable container fitted with a stirrer. The powder blend was placed in a double sigma blender and was mixed first with 21 g of soya lectin and then with the povidone solution until a mass with a suitable appearance and plasticity was obtained. Total mixing time: 4 minutes. The mixture was screened in a wet granulator fitted with a 3 mm mesh screen. The resulting granulate was dried in a fluid-bed drier at a temperature of 40° C. for 2 hours. Finally, the dry granulate was sieved through an oscillating sieve with a 1.4 mm mesh and then through a vibrating sieve with a 0.355 mm mesh to separate the fine fraction. The fine fraction was discarded. The friability of the granulate with a particle size comprised between 1.4 and 0.355 mm is 0.20%.

### b) Coating of the Granules:

In order to prepare the coating solution, 150 g of ethylcellulose were added to an isopropanol (1200 ml)/acetone (1800 ml) mixture in a glass container fitted with an anti-combustion stirrer. Then the solution was sieved through a 0.1 mm mesh sieve, stirring gently and constantly throughout the process. The screened granule was placed in a fluid bed coater in order to coat it with the ethylcellulose solution. The weight increase after the coating process was 14.04%.

### c) Compression:

795.2 g of microcrystalline cellulose PH 101, 186.4 g of microcrystalline cellulose PH 102 and 23.3 g of magnesium stearate were added to 1092 g of the coated granulate. The resulting mixture was blended in a double-cone blender at 25 rpm for 5 minutes, and then compressed using oval punches in order to obtain tablets with a weight of 700 mg.

### d) Coating of the Tablets:

The tablets were coated with Sepifilm® 752 white in a coating pan until a 1.5% weight increase was achieved. The dissolution profile is given below:

| Time (h) | % Dissolved |
|---|---|
| 1 | 1.7 |
| 4 | 9.1 |
| 8 | 20.1 |
| 20 | 50.8 |

### Example 3

| | Ingredient | Amount (mg) |
|---|---|---|
| Granules | Metoprolol succinate | 190.00 |
| | (47.9% w/w of granule) | |
| | Microcrystalline cellulose PH 101 | 94.60 |
| | Methylcellulose | 95.00 |
| | Maize starch | 15.50 |
| | Glycerol | 1.90 |
| | Granules | 397.00 |
| Coated granules | Granules to be coated | 378.00 |
| | Ethylcellulose N100 | 63.8 |
| | Coated granules | 441.80 |
| Tablet | Coated granules (equivalent to | 441.80 |
| | 180.5 mg of metoprolol succinate) | |
| | Uncoated granules (equivalent to | 19.00 |
| | 9.5 mg of metoprolol succinate) | |
| | Microcrystalline cellulose PH 101 | 524.20 |
| | Magnesium stearate | 15.00 |
| | Tablet | 1000.00 |
| Coated tablet | Film coating suspension: | 15.00 |
| | Sepifilm ® 752 white | |
| | Coated tablet | 1015.00 |

### Method of Preparation:

Batch size: 5200 tablets

### a) Granulation:

1027.5 g of metoprolol succinate, 512 g of microcrystalline cellulose PH 101 and 514 g of methylcellulose were sieved through a 2 mm mesh screen. The screened components were placed into a mixer and mixed for 2 minutes at 200 rpm. Separately, a starch paste was prepared in a suitable glass or stainless steel container. 84 g of maize starch and 10.5 g of glycerol were added to 1195 ml of purified water with the impeller in motion. The mixture was heated with constant stirring until 80-85° C. Once this temperature was reached the mixture was allowed to cool to room temperature (25-30° C.) under constant stirring. The maize starch paste should have a viscous appearance.

The resulting maize starch paste was transferred to the blender/kneader and then it was kneaded for 2 minutes at an impeller speed of 200 rpm without the chopper and then a further 2 minutes with the chopper at 100 rpm. The mixture was screened in a wet granulator fitted with a 5 mm mesh screen. The resulting granulate was transferred to the fluid bed drier and was dried at 40° C. for 2 hours. The water content of the dry granulate was checked to be lower than 2.5% w/w. The dry granulate was screened through a centrifugal granulator fitted with a 1.5 mm mesh screen and then through a vibrating sieve with a 0.355 mm mesh to separate the fine fraction. The friability of the granulate with a particle size comprised between 1.5 and 0.355 mm is 0.25%.

### b) Coating of the Granules:

In a suitable stainless steel container fitted with a pneumatic anti-combustion stirrer that contained 2840 ml of isopropanol and 4260 ml of acetone, 355 g of ethylcellulose N-100 were dissolved and it was checked for complete dissolution after 2 hours stirring. Once the ethylcellulose was completely dissolved, the solution was filtered through a 0.25 mm mesh screen and it was collected in a suitable container. The filtered solution was diluted to compensate for loss through evaporation of the solvents during handling. The granulate was placed in fluid bed equipment and coated with this solution. The weight increase after the coating process was 16.88%.

### c) Compression:

2726 g of microcrystalline cellulose PH 101 and 78 g of magnesium stearate were separately sieved though a 0.6 mm mesh screen. 2297 g of the coated granulate, 99 g of the uncoated granulate (the fraction of granulate with <0.355 mm particle size separated at the end of the granulation step) and the microcrystalline cellulose PH 101 were blended in a double-cone blender for 15 minutes at 25 rpm. Then the magnesium stearate was added and blended for a further 5 minutes. The mixture was compressed using oval punches in order to obtain tablets with a weight of 1000 mg.

### d) Coating of the Tablets:

The tablets were coated with Sepifilm® 752 white in a coating pan until a 1.5% weight increase was achieved. The dissolution profile is given below:

| Time (h) | % Dissolved |
|---|---|
| 1 | 18.0 |
| 4 | 33.7 |
| 8 | 50.2 |
| 20 | 79.4 |

### Example 4

| | Ingredient | Amount (mg) |
|---|---|---|
| Granules | Tolterodine tartrate | 4.00 |
| | Microcrystalline cellulose PH 101 | 6.00 |
| | Methylcellulose | 6.10 |
| | Maize starch | 2.10 |
| | Glycerol | 0.25 |
| | Granules | 18.45 |
| Coated granules | Granules to be coated | 18.45 |
| | Ethylcellulose N100 | 3.15 |
| | Coated granules | 21.60 |
| | Microcrystalline cellulose PH 101 | 76.40 |
| | Magnesium stearate | 2.00 |
| | Tablet | 100.00 |
| Coated tablet | Film coating suspension: | 3.00 |
| | Sepifilm ® 770LP | |
| | Coated tablet | 103.00 |

Granules are prepared and coated following the procedure of Example 3 and adjusting the quantities of the excipients to the formula above. By using other proportions of ethylcellulose in the coating, the release profiles of extended release granules can be modified. The granules can be included in tablets, as in this example, with the addition of diluents and lubricants and then subsequently compressed or filled into capsules or sachets. The percentage of tolterodine tartrate used in the granules can be modified and/or the percentage of diluent used for instance in the preparation of tablets can be modified and/or the weight of the tablets be modified in forms known by the person skilled in the art.

### Example 5

| | Ingredient | Amount (mg) |
|---|---|---|
| Granules | Quetiapine fumarate | 230.26 |
| | Microcrystalline cellulose PH 101 | 77.50 |
| | Methylcellulose | 78.00 |
| | Maize starch | 12.70 |
| | Glycerol | 1.54 |
| | Granules | 400.00 |
| Coated granules | Granules to be coated | 400.00 |
| | Ethylcellulose N100 | 68.00 |
| | Coated granules | 468.00 |
| | Microcrystalline cellulose PH 101 | 517.00 |
| | Magnesium stearate | 15.00 |
| | Tablet | 1000.00 |
| Coated tablet | Film coating suspension: | 30.00 |
| | Sepifilm ® 770LP | |
| | Coated tablet | 1030.00 |

Granules are prepared and coated following the procedure of Example 3 replacing metoprolol succinate by quetiapine hemifumarate and adjusting the quantities of the excipients to the formula above. By using other proportions of ethylcellulose in the coating, the release profiles of extended release granules can be modified. In the case of quetiapine hemifumarate, the proportion of ethylcellulose can be reduced due to the lower solubility of this salt to achieve a less pronounced extended effect over time. The granules can be included in tablets, as in this example, with the addition of diluents and lubricants or filled into capsules or sachets.

### Example 6

### Comparative Example

Granules were prepared having the same composition as that of the pellets described in Example 15 of WO 2006048895-A1.

| | |
|---|---|
| Metoprolol succinate | 800.0 g |
| Microcrystalline cellulose (Avicel PH 101) | 200.0 g |
| Povidone K 29-32 | 10.0 g |

800 g of metoprolol succinate and 200 g of microcrystalline cellulose were sieved through a mesh of 0.8 mm. The screened components were placed into a mixer and mixed for 20 minutes at 20 rpm. A solution of Povidone K 29-32 was prepared by dissolving 10 g in 100 ml of demineralised water. The previous mixture is kneaded with this solution and 100 ml of demineralized water added to achieve a suitable consistency of the mass. The mixture was screened in a wet granulator fitted with a 3 mm mesh screen. The resulting granulate was transferred to the fluid bed drier and was dried at 40° C. for 90 minutes. The water content of the dry granulate was 1.08%. The dry granulate was screened through a centrifugal granulator fitted with a 2 mm mesh screen and then through a vibrating sieve with a 0.2 mm mesh to separate the fine fraction. The friability of the granulate with a particle size comprised between 0.2 and 2 mm is 42.6%. Therefore these granules are not suitable to proceed with the coating process required by the invention.

1. An extended release coated granule comprising a granule having a particle size ranging from 0.2 to 2 mm, a friability lower than or equal to 1% and comprising metoprolol succinate as active ingredient in an amount ranging from 10 to 75% by weight of the granule and at least one binder selected from one or both of microcrystalline cellulose and methylcellulose, said granule being coated with a film-former coating agent.

2. (canceled)

3. The coated granule according to claim 1, wherein the granule has a particle size ranging from 0.2 to 1 mm and a friability lower than 1%.

4. The coated granule according to claim 1, wherein the amount of metoprolol succinate in the granule ranges from 40 to 75% by weight of the granule, and the friability of the granule is lower than 1%.

5. (canceled)

6. The coated granule according to claim 1, wherein microcrystalline cellulose and methylcellulose are used as binders.

7. The coated granule according to claim 1, further comprising one or more pharmaceutically acceptable ingredients selected from the group consisting of starch; maize starch; gelatin; povidones; arabic gum; tragacanth gum; pectin; dextrin; glyceryl behenate; alginates; mannitol; lactose; hydroxyethylcellulose and its derivatives; hydroxyethylmethylcellulose and its derivatives; hydroxypropylcellulose and its derivatives; hydroxypropylmethylcellulose and its derivatives; bicalcium phosphate; tricalcium phosphate; lactose-povidone complexes; lactose-colloidal silica dioxide; liposaccharide-alkaline earth orthophosphate salt complexes; calcium carbonate and its derivatives; and calcium carbonate co-processed mixtures of calcium carbonate with sorbitol, mannitol, any other kind of saccharides, polysaccharides, copolyvidones, dextrins, maltodextrins, carboxymethylcelluloses, pregelatinized starch, cyclodextrins, cellulose ethers, calcium gluconates, or calcium gluconates-lactates.

8. (canceled)

9. The coated granule according to claim 1, wherein the film-former coating agent is selected from the group consisting of: ethylcellulose; mono-, di- or triglycerides; fatty acids; waxes; synthetic mixed glycerides; hydrophilic cellulose derivatives with medium or high viscosity; polyvinyl acetates and chlorides; calcium phosphates and sulphates; hydrocolloids, hydrogels, methacrylic polymer compounds and derivatives; cellulose aceto-phthalates; cellulose hydrogen phthalates; and alginic acid derivatives.

10. (canceled)

11. A process for the preparation of extended release coated granules as defined in claim 1 comprising the steps of:
a) granulating a mixture comprising metoprolol succinate and at least one binder selected from one or both of microcrystalline cellulose and methylcellulose, wherein the resulting amount of metoprolol succinate in the dry granules is comprised between 10 and 75% by weight;
b) drying the granules resulting from step (a) if required;
c) sieving the dried granules through a sieve with a mesh size of 1 to 2 mm; and then through a sieve with a mesh size of 0.2 to 0.4 mm in order to separate the granules with a size lower than the mesh size used; and
d) coating the dried granules resulting from step (c) with a dispersion of a film-former coating agent.

12. The process according to claim 11, wherein the granulation of step (a) further comprises the addition of a binding solution comprising at least one binder, the binder solution including a solution of maize starch in a mixture of glycerol and water.

13. The process according to claim 11, wherein the coating of step (d) is carried out using an amount of film-former coating agent ranging from 1 to 20% by weight in an appropriate solvent system resulting in a weight increase of between 10 and 40%; using fluid bed equipment.

14. The process according to claim 11, wherein the coating of step (d) results in a weight increase of the granule of between 20 and 30%.

15. (canceled)

16. (canceled)

17. (canceled)

18. The process according to claim 11, wherein the film-former coating agent is dissolved in a solvent selected from the group consisting of ethanol, acetone, isopropylic alcohol, methylene chloride, water and mixtures of any two or more thereof.

19. An extended release pharmaceutical composition comprising coated granules according to claim 1 together with appropriate amounts of pharmaceutical excipients or carriers.

20. The extended release pharmaceutical composition according to claim 19, comprising at least 90% by weight of metoprolol succinate as coated granules and up to 10% by weight of metoprolol succinate as uncoated granules with a particle size not greater than 0.4 mm, together with appropriate amounts of pharmaceutical excipients or carriers.

21. The extended release pharmaceutical composition according to claim 20 comprising at least 95% by weight of metoprolol succinate as coated granules and up to 5% by weight of metoprolol succinate as uncoated granules.

22. (canceled)

23. (canceled)

24. (canceled)

25. (canceled)

26. (canceled)

27. The extended release pharmaceutical composition according to claim 19, wherein the amount of metoprolol succinate in the granule ranges from 40 to 75% by weight of granule, and the friability of the granule is lower than 1%.

28. The extended release pharmaceutical composition according to claim 19, wherein the granule further comprises one or more pharmaceutically acceptable ingredients selected from the group consisting of starch; maize starch; gelatin; povidones; arabic gum; tragacanth gum; pectin; dextrin; glyceryl behenate; alginates; mannitol; lactose; hydroxyethylcellulose and its derivatives; hydroxyethylmethylcellulose and its derivatives; hydroxypropylcellulose and its derivatives; hydroxypropylmethylcellulose and its derivatives; bicalcium phosphate; tricalcium phosphate; lactose-povidone complexes; lactose-colloidal silica dioxide; liposaccharide-alkaline earth orthophosphate salt complexes; calcium carbonate and its derivatives; and calcium carbonate co-processed mixtures of calcium carbonate with sorbitol, mannitol, any other kind of saccharides, polysaccharides, copolyvidones, dextrins, maltodextrins, carboxymethylcelluloses, pregelatinized starch, cyclodextrins, cellulose ethers, calcium gluconates, or calcium gluconates-lactates.

29. The extended release pharmaceutical composition according to claim 27, which is in the form of a tablet.

30. The extended release pharmaceutical composition according to claim 21, which is in the form of a tablet.

31. The extended release pharmaceutical composition according to claim 19 in the form of tablets comprising coated granules, wherein the granules of said coated granules comprise metoprolol succinate in an amount ranging from 40 to 60% by weight of the granule, at least one binder selected from microcrystalline cellulose and methylcellulose, and starch in a amount equal to or less than 3.90% by weight of granule, the granules having a particle size distribution ranging form 0.2 to 1 mm, and are coated with a film-former coating agent.
